(19) United States
(12) Patent Application Publication
(10) Pub. No.: US 2024/0158462 A1
(43) Pub. Date: May 16, 2024

(54) REPORTER CELLS EXPRESSING CHIMERIC POLYPEPTIDES FOR USE IN DETERMINING PRESENCE AND OR ACTIVITY OF IMMUNE CHECKPOINT MOLECULES

(71) Applicant: B.G. Negev Technologies & Applications Ltd., at Ben-Gurion University, Beer-Sheva (IL)

(72) Inventors: Moshe ELKABETS, Beit Kama (IL); Angel PORGADOR, LeHavim (IL)

(73) Assignee: B.G. Negev Technologies & Applications Ltd., at Ben-Gurion University, Beer-Sheva (IL)

(21) Appl. No.: 18/244,259

(22) Filed: Sep. 10, 2023

Related U.S. Application Data
(63) Continuation of application No. PCT/IL2022/050268, filed on Mar. 9, 2022.
(60) Provisional application No. 63/159,072, filed on Mar. 10, 2021.

(51) Int. Cl. C07K 14/705; C07K 14/725; G01N 33/50

(52) U.S. Cl. CPC C07K 14/70521; C07K 14/7051; C07K 14/70532; G01N 33/5041; C07K 2319/03; C07K 2319/74

(57) ABSTRACT

A polynucleotide is provided. The polynucleotide encoding a chimeric polypeptide comprising an amino acid sequence of an immune checkpoint molecule capable of binding a ligand thereof, the immune checkpoint molecule being translationally fused to another amino acid sequence of a cell signaling module such that upon binding of the immune checkpoint molecule to the ligand, the cell signaling module is activated. Also provided other configurations of the polynucleotide, cells comprising the polynucleotides and methods of using the cells expressing the polynucleotides.

### RELATED APPLICATIONS

This Application is a Continuation of PCT Patent Application No. PCT/IL2022/050268 having International filing date of Mar. 9, 2022, which claims the benefit of priority under 35 USC § 119(e) of U.S. Provisional Patent Application No. 63/159,072 filed on Mar. 10, 2021. The contents of the above applications are all incorporated by reference as if fully set forth herein in their entirety.

### SEQUENCE LISTING STATEMENT

The XML file, entitled 97628SecondReplacementSequenceListing.xml, created on Dec. 19, 2023, comprising 60,107 bytes, is incorporated herein by reference.

### FIELD AND BACKGROUND OF THE INVENTION

The present invention, in some embodiments thereof, relates to reporter cells expressing chimeric polypeptides for use in determining presence and or activity of immune checkpoint molecules or their ligands.

Immune checkpoint blockers (ICBs, BOX1) have shown remarkable positive outcome as a treatment modality in medical oncology ultimately prolonging the survival of a fraction of cancer patients. ICBs are mainly antibody-based drugs that activate T cells killing via blocking suppressive immunomodulatory proteins like programmed cell death protein 1 (PD-1) or cytotoxic T lymphocyte-associated protein 4 (CTLA-4) that can shrink tumors and cure patients. The major problems associated with treating cancer patients with ICBs are (i) only 5-40% of patients respond to ICBs, (ii) induction of severe aberrant effects and auto-immune reactions, (iii) costly treatment.

Thus, knowing upfront who will respond to ICBs, will increase the response rate of ICBs and spare ineffective treatments. Altogether tailoring a personalized immunotherapy treatment is an urgent unmet clinical need that will save cancer patients' lives and improve their quality of life.

In the last decade, an intensive research effort was directed toward identifying biomarkers of response to ICB. Currently, measuring immunomodulatory proteins of PD-L1 by immune histochemistry (IHC) improves the prediction of response to anti-PD1/PD-L1 therapies, and genomic sequencing and calculation of tumor mutational burden (TMB), or microsatellite instability expression improve the prediction to anti-PD1 to about ˜30-50%. Another biomarker of positive response to ICB is the association with massive infiltration of lymphocytes defined as “hot” tumors, while “cold” tumors (with low T cell infiltrations) are less responsive to ICBs. Tumors that do not respond to ICBs carry at least a single innate resistance mechanisms (Kalbasi and Ribas Nat. Immunol. Rev. 2020 January; 20 (1):25-39), while the common resistance mechanisms are (i) reduction of tumor cells immunogenicity by downregulating MHC class-I expression and low presentation of a tumor antigen or neoantigen, (ii) upregulation of immunosuppressive immunomodulators like PD-L1, CTLA-4, TIGIT, TIM3 etc., (iii) presence and accumulation of immune and stromal cells in the tumor microenvironment (TME) like different subtypes of CD4+ and CD8+ T-cells (Huang 2020 Font. Cell Dev. Biol. January; 20 (1):25-39), B-cells, myeloid cells, dendritic cells, and cancer-associated fibroblasts.

Identification of biomarkers/predictors of response can be efficiently developed by analyzing omics data from cancer patients. Specifically, a meta-analysis of gene expression has enabled numerous insights into biological systems that gain statistical power and increase the signal-to-noise ratio to overcome the biases of individual studies. Such an approach has been used to uncover disease subtypes, to predict survival, and to discover biomarkers and therapeutic targets (Auslander et al. 2020 Mol. Syst. Biol. December; 16 (12):e9701). Moreover, recently, transcriptomics data was shown to be informative in predicting the response to anti-PD-1 or CTLA-4 in melanoma and provided new knowledge of immunomodulators that limit immunotherapy in this type of cancer (Auslander et al. Nat. Med. 2018 October; 24 (10):1545-1549).

Currently, measurement of the immunomodulatory proteins level is primarily assessed by IHC staining of the tumors. Although this approach is well-validated, a few drawbacks exist: (1) IHC staining is a multistep process that takes a few days and requires a pathologist; While the evaluation is reliable, a variation between the IHC scoring exists among the pathologists; (2) IHC detects expression levels of the protein; however, quantification of the suppression activity of the immunomodulatory proteins is currently unmeasurable; (3) A single immunomodulatory receptor can bind to several ligands (i.e., LAG3 has 3 different ligands that induce a negative signal in T cells), and measuring all ligands in tissue is challenging and almost unfeasible.

### SUMMARY OF THE INVENTION

According to an aspect of some embodiments of the present invention there is provided a polynucleotide encoding a chimeric polypeptide comprising an amino acid sequence of an immune checkpoint molecule capable of binding a ligand thereof, the immune checkpoint molecule being translationally fused to another amino acid sequence of a cell signaling module such that upon binding of the immune checkpoint molecule to the ligand, the cell signaling module is activated.

According to an aspect of some embodiments of the present invention there is provided a polynucleotide encoding a chimeric polypeptide comprising an amino acid sequence of an immune checkpoint molecule capable of binding a receptor thereof, the immune checkpoint molecule being translationally fused to another amino acid sequence of a cell signaling module such that upon binding of the immune checkpoint molecule to the receptor, the cell signaling module is activated.

According to an aspect of some embodiments of the present invention there is provided a nucleic acid expression construct comprising a nucleic acid sequence encoding the polynucleotide under transcriptional control of a cis-acting regulatory element(s).

According to an aspect of some embodiments of the present invention there is provided a reporter cell comprising the polynucleotide or the nucleic acid construct.

According to an aspect of some embodiments of the present invention there is provided a method of detecting presence and/or activity of a ligand of an immune checkpoint molecule in a cancer cell or a cell in a microenvironment of the cancer cell, the method comprising:
(a) contacting the cancer cell with the above-mentioned reporter cell;
(b) determining activation of the cell signaling module in the reporter cell, the activation being indicative of the presence and/or activity of the ligand of the immune checkpoint molecule in the cancer cell or cell in the microenvironment.

According to an aspect of some embodiments of the present invention there is provided a method of detecting presence and/or activity of a receptor of an immune checkpoint molecule in an immune cell, the method comprising:
(a) contacting the immune cell with the reporter cell;
(b) determining activation of the cell signaling module in the reporter cell, the activation being indicative of the presence and/or activity of the receptor of the immune checkpoint molecule in the immune cell.

According to an aspect of some embodiments of the present invention there is provided a method of treating a subject diagnosed with cancer, the method comprising:
(a) contacting the cancer cell or a cell in a microenvironment of the cancer cell of the subject with the reporter cell;
(b) determining activation of the cell signaling module in the reporter cell, the activation being indicative of the presence and/or activity of the ligand of the immune checkpoint molecule in the cancer cell or the cell in the microenvironment of the cancer cell; and
(c) treating the subject with a modulator of the immune checkpoint molecule when presence or a predetermined threshold of activity of the ligand of the immune checkpoint molecule is indicated or with another treatment modality when it is not indicated or absent.

According to an aspect of some embodiments of the present invention there is provided a method of selecting treatment for a subject diagnosed with cancer, the method comprising:
(a) contacting the cancer cell or a cell in a microenvironment of the cancer cell of the subject with the reporter cell;
(b) determining activation of the cell signaling module in the reporter cell, the activation being indicative of the presence and/or activity of the ligand of the immune checkpoint molecule in the cancer cell or the cell in the microenvironment of the cancer cell; and
(c) selecting treatment for the subject with a modulator of the immune checkpoint molecule when presence or a predetermined threshold of activity of the ligand of the immune checkpoint molecule is indicated or with another treatment modality when it is not indicated or absent.

According to some embodiments of the invention, there is provided the chimeric polypeptide encoded by the polynucleotide.

According to some embodiments of the invention, the immune checkpoint molecule is selected from the group consisting of CTLA4, PD-1, LAG3, TIGIT, TIM3, VISTA, CEACAM1, CD28, OX40, CD137(4-1BB), GITR, ICOS, CD27, CD80, CD86, PD-L1, PD-L2, MHC class II/lectins, CD155, Galectin 9, VSIG-3, B7, CD80, CD86, OX40L, CD137L, GITRL, ICOSLG and CD70.

According to some embodiments of the invention, the immune checkpoint molecule is PD-1.

According to some embodiments of the invention, the immune checkpoint molecule is CTLA4.

According to some embodiments of the invention, the immune checkpoint molecule is naturally expressed on an immune cell and wherein the ligand is naturally expressed on a cancer cell.

According to some embodiments of the invention, the immune checkpoint molecule is naturally expressed on a cancer cell and wherein the ligand is naturally expressed on an immune cell.

According to some embodiments of the invention, the cell signaling module comprises a transmembrane domain and/or a cytoplasmic portion of a cell signaling receptor.

According to some embodiments of the invention, the cell signaling module comprises a transmembrane domain and/or a cytoplasmic portion of a receptor kinase.

According to some embodiments of the invention, the receptor kinase is a tyrosine kinase or serine/threonine kinase.

According to some embodiments of the invention, the cell signaling module comprises an adaptor molecule.

According to some embodiments of the invention, the cell signaling module comprises a CD3 zeta chain.

According to some embodiments of the invention, activation of the cell signaling module is by dimerization, oligomerization and/or post-translational modification.

According to some embodiments of the invention, the determining activation is by analyzing a cytokine and/or an interleukin induced by the activation.

According to some embodiments of the invention, the interleukin is selected from the group consisting of IL-2 and IL-8.

According to some embodiments of the invention, the determining activation is by analyzing a phenotype selected from the group consisting of proliferation, apoptosis, migration, post-translational modification, biomolecule expression, biomolecule secretion, morphology and cell cycle distribution.

According to some embodiments of the invention, the cell is an immune cell.

According to some embodiments of the invention, the cell is a non-cancerous cell.

According to some embodiments of the invention, the cell is a transgenic cell.

According to some embodiments of the invention, the cell is transformed to express a fluorescent or bioluminescent molecule upon activation of the cell signaling module.

According to some embodiments of the invention, the contacting is in the presence of an immune checkpoint modulator.

According to some embodiments of the invention, the immune checkpoint modulator is an anti PD-1 antibody.

According to some embodiments of the invention, the cancer cell is comprised in a tissue biopsy.

According to some embodiments of the invention, the tissue biopsy is fresh.

According to some embodiments of the invention, the tissue biopsy is fixated.

According to some embodiments of the invention, the contacting is with a plurality of the chimeric polypeptides of different immune checkpoint molecules sequentially or simultaneously.

According to some embodiments of the invention, the method further comprises contacting the cancer cells with interferon gamma to induce expression of immune checkpoint molecule.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show expression level of IcAR-PD-1 in a transfected cell line and response to anti-PD-1. FIG. 1A—IcAR-PD-1 present high expression level of PD-1 FIG. 1B—IcAR-PD-1 response to anti-PD-1.

FIGS. 2A-C show IcAR-PD-1 response to cells overexpressing PD-L1 in the presence or absence of anti-PD-L1. FIG. 2A—IcAR-PD-1 response correlates (R²=0.9868) with A549 PD-L1 expression. PD-L1 expression of A549 was manipulated by pretreatment with titrated amounts of IFNγ for 24 hrs before incubation with the IcAR. FIG. 2B. IcAR-PD-1 and A549 calibration experiments shows that 3100 A549 cells are sufficient to provide a strong signal. FIG. 2C. IcAR-PD-1 response to PD-L1 is blocked by anti PD-L1 (Durvalumab).

FIG. 3 shows IcAR-PD-1 activation by tumors with different levels of PD-L1 expression.

FIGS. 4A-B show an IcAR assay on fixated tissue samples. FIG. 4A—IHC staining of HNC tumors. FIG. 4B—Production of IL-2 from IcAR-PD-1 on FFPE samples, as in FIG. 4A.

FIGS. 5A-B show the prediction of response to PD1/PD-L1 treatment using patient-derived FFPE cuts. FIG. 5A—IcAR score derived by PD-1 blockade using pembrolizumab show significant correlation between clinical response to IcAR score (Spearman R=0.8913, p<0.0001) with medium linear regression (R²=0.6248, p<0.0001). FIG. 5B—IcAR score derived by PD-L1 blockade using durvalumab shows significant correlation between clinical response to IcAR score (Spearman R=0.8989, p<0.0001) and strong linear regression (R²=0.8474, p<0.0001).

FIGS. 6A-C show expression of PD-1 ligands: PD-L1 and PD-L2. FIG. 6A—shows analysis of PD-L1 expression on the surface of cells harvested from patient-derived xenografts (PDX), while FIG. 6B shows PD-L2 expression on the same PDX samples. FIG. 6C—A549 shows low expression of PD-L1 and PD-L2 in the control group, while incubations of cells with the cytokine IFN-ginduces high levels levels of PD-L2 and PD-L2. Blocking the receptors and the ligands show that IcAR-PD-1 can recognize both ligands.

FIG. 7 shows prediction of response to CTLA4 treatment using patient-derived FFPE cuts. In a sample (n=12) of PD-1/PD-L1 blockade treated patients, there was no correlation between IcAR score and response to treatment.

FIG. 8 shows the expression of different immune-checkpoint (IC) ligands. Different PDXs show variation in expression of different IC ligands. For example, LSE16 (black) exhibit high levels of CD155 (TIGIT ligand), CD66 (TIM3 ligand) but does not express CD80 (CTLA4 ligand).

FIG. 9 compares IcAR-PD1 scoring in normal and colon cancer tissues. IcAR-PD1 cells were co-cultured with four samples of colon cancer. Two of the tissue samples were analyzed with approximate normal tissues of the colon. Data shows that normal tissues received a negative score while cancer tissues received a positive score.

FIG. 10 shows IL-2 expression upon co-culture of IcAR-TIGIT with 7 cancer tissues obtained from colon cancer patients. One of the sample showed very high levels of IL-2, which indicates high amounts of TIGIT's ligands.

FIG. 11 is a scheme of a Lentivirus expression vector according to some embodiments of the invention.

### DESCRIPTION OF SPECIFIC EMBODIMENTS OF THE INVENTION

The present invention, in some embodiments thereof, relates to reporter cells expressing chimeric polypeptides for use in determining presence and or activity of immune checkpoint molecules or their ligands.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Treatment of cancer patients with immune checkpoint blockers (ICBs) revolutionized medical oncology but with it new challenges arose. Among such challenges are autoimmunity toxicity and high costs associated with the treatment. These concerns are expected to be amplified when more FDA-approved ICBs enter routine medical practice. Intensive research is performed to overcome such challenges, including predicting the response to ICBs. Currently, there are several biomarkers for response to anti-PD1/PD-L1 therapies, which improve the prediction to about 50%. These include, PD-L1 expression in lung cancer patients, MMR levels, or tumor burden mutation status in the colon cancer and stomach cancer. However, in most cancers, there are no predictive markers, resulting in over 80% of cancer patients that receive ineffective ICBs treatment and suffer from unnecessary toxicity.

Whilst conceiving embodiments of the invention and reducing it to practice, the present inventors configured a cell-based reporter system that can recognize immunosuppressive ligands that block immune cell activation. In one embodiment, this system is referred to as “Immuno-Check point Artificial Reporter (IcAR)”. The PD-1 IcAR recognizes PD-L1 availability with high specificity and sensitivity on fresh and formalin-fixed paraffin-embedded (FFPE) tissues. The present inventors were able to establish the IcAR assay over a plurality of reporter systems in which the CTLA4 and TIGIT were employed as the immune checkpoint molecule arms (Example 7 and Example 10, respectively).

The present inventors have shown that the IcAR test correlates with clinical responsiveness in a retrospect study (Example 6). They further showed that the assay may be augmented by implementing the IcAR assay on normal samples (non-cancerous) from a matching tissue to decipher the background score level and to predict toxicity of treatment (Example 9).

It is expected that the newly devised reporter system will have significant contribution to cancer patients, such that measuring immunomodulators' activity becomes standard for predicting response to immune checkpoint modulation.

Thus, according to an aspect of the invention there is provided a polynucleotide encoding a chimeric polypeptide comprising an amino acid sequence of an immune checkpoint molecule capable of binding a ligand thereof, the immune checkpoint molecule being translationally fused to another amino acid sequence of a cell signaling module such that upon binding of the immune checkpoint molecule to the ligand, the cell signaling module is activated.

According to another aspect of the invention there is provided a polynucleotide encoding a chimeric polypeptide comprising an amino acid sequence of an immune checkpoint molecule capable of binding a receptor thereof, the immune checkpoint molecule being translationally fused to another amino acid sequence of a cell signaling module such that upon binding of the immune checkpoint molecule to the receptor, the cell signaling module is activated.

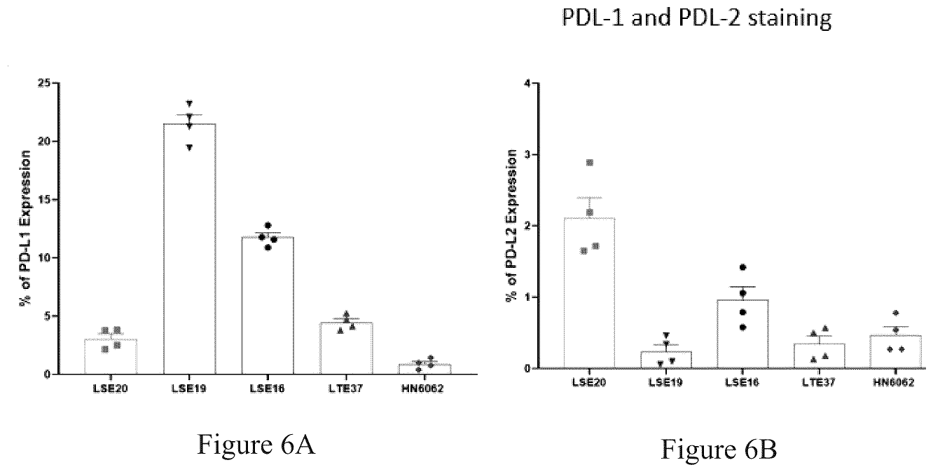

It will be appreciated that according to some embodiments, especially when there is more than one ligand to a specific immune checkpoint receptor (e.g., in the case of PD-1) or more receptors to a specific ligand, the use of both chimeric molecules (one including the receptor and one including a ligand) is contemplated, wherein a difference in activity of the signaling molecule may infer activity of more than one player in the cancerous tissue (see for instance FIGS. 6A-C).

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above). According to a specific embodiment, the polynucleotide is dsDNA.

The term “isolated” refers to at least partially separated from the natural environment e.g., from a plant cell.

As used herein “a chimeric polypeptide” or “fusion polypeptide” refers to a polypeptide in which proteinaceous components which are not found in nature on a single polypeptide or at the same orientation on a single polypeptide are fused, typically covalently and preferably by a peptide bond. Thus the proteinaceous components are heterologous to one another.

As used herein, the term “heterologous” refers to an amino acid sequence which is not native to the recited amino acid sequence at least in localization or is completely absent from the native sequence of the recited amino acid sequence.

The components can be linked directly or via a linker (e.g., amino acid linker).

Non-limiting examples of polypeptide linkers include linkers having the sequence LE, GGGGS (SEQ ID NO: 1), (GGGGS)ₙ (n=1-4) (SEQ ID NO: 2), GGGGSGGGG (SEQ ID NO: 3), (GGGGS)×2 (SEQ ID NO: 4), (GGGGS)×2+GGGG (SEQ ID NO: 5), (Gly)₈ (SEQ ID NO: 6), (Gly)₆(SEQ ID NO: 7), (EAAAK)ₙ (n=1-3) (SEQ ID NO: 8), A(EAAAK)ₙA (n=2-5) (SEQ ID NO: 9), AEAAAKEAAAKA (SEQ ID NO: 10), A(EAAAK)₄ALEA(EAAAK)4A (SEQ ID NO: 11), PAPAP (SEQ ID NO: 12), KESGSVSSEQ LAQFRSLD (SEQ ID NO: 13), EGKSSGSGSESKST (SEQ ID NO: 14), GSAGSAAGSGEF (SEQ ID NO: 15), and (XP)ₙ, with X designating any amino acid, e.g., Ala, Lys, or Glu.

As used herein, the terms “immune checkpoint,” “checkpoint pathway,” and “immune checkpoint pathway” refer to a pathway by which the binding of an immune checkpoint ligand to an immune checkpoint receptor modulates the amplitude and quality of the activation of immune cells (e.g., T cells, Jurkat cells, HuT-78, CEM, Molt-4, etc.).

As used herein “an immune checkpoint molecule” refers to at least the portion of an immune checkpoint molecule that is capable of binding a ligand thereof which modulates its activity. It is typically an immune checkpoint receptor. These immune checkpoint molecules are regulatory molecules that maintain immune homeostasis in physiological conditions. By sending T cells a series of co-stimulatory or co-inhibitory signals via receptors, immune checkpoints can both protect healthy tissues from adaptive immune response and activate lymphocytes to remove pathogens effectively. However, due to their mode of action, suppressive immune checkpoints serve as unwanted protection for cancer cells.

According to a specific embodiment, the immune checkpoint molecule is of an immune cell (e.g., PD-1) and the ligand is of a cancer cell (e.g., PD-L1).

As used herein, the term “ligand of an immune checkpoint molecule” or “immune checkpoint ligand” (“ICL”) refers to a ligand of an immune checkpoint receptor “Immune checkpoint ligands” are commonly surface-displayed proteins on antigen presenting cells (APCs) or tumor cells. Through an interaction with an immune-cell-displayed immune checkpoint receptor, an “immune checkpoint ligand” modulates the immune response of the immune cell (e.g., T cell) to the antigen presenting cell. Examples of “immune checkpoint ligands” that bind inhibitory immune checkpoint receptors include, but are not limited to, PD-L1, PD-L2, B7-H4, CD 155, galectin-9, HVEM, etc.

However, the terminology may be the other way around, as both ligand and receptor are typically membrane-anchored.

Examples of immune checkpoint molecules and their ligands that are contemplated according to some embodiments of the present invention are provided herein below in Table 1.

TABLE 1*
Examples of suppressive (negative) and stimulatory (positive)
immune checkpoint ligand-receptor pairs with cellular distribution
of these molecules under physiological conditions

| | Cellular Distribution of Immune Checkpoint | | |
|---|---|---|---|
| | the Ligand Expression Receptor | | Cellular Expression of |
| Ligand | Suppressive (negative) immune checkpoints | | the Receptor Expression |
| CD80 or CD86 | Antigen-presenting cells | CTLA4 | Activated T cells, Tregs |
| PD-L1 (CD274) or | DCs, macrophages, peripheral | PD-1 | Activated B and T cells, |
| PD-L2 (CD273) | non-lymphoid tissues | | APCs, NK cells |
| MHC class | Antigen-presenting cells | LAG3 | Activated T cells, |
| II/Lectins | | | Tregs, NK cells, B |
| | | | cells, DCs |
| CD155/CD112 | Normal epithelial, | TIGIT | Activated T cells, |
| | endothelial, neuronal, and | | Tregs, NK cells |
| | fibroblastic cells | | |
| Galectin | Multiple tissues | TIM3 | Activated T cells |
| 9/PtdSer/HMGB1 | | | |
| VSIG-3 | Neurons and glial cells | VISTA | Naïve and activated T |
| | | | cells |
| CEACAM1 | T and NK cells | CEACAM1 | Activated T and NK |
| | | | cells |
| B7 molecules: | Antigen-presenting cells | CD28 | T cells |
| CD80 or CD86 | | | |
| OX40L | DCs, macrophages, B | OX40 | Activated T cells, Tregs, |
| | cells, endothelial cells, | | NK cells, neutrophils |
| | smooth muscle cells | | |
| CD137L | Antigen-presenting cells | CD137 | Activated Tcells, NK |
| | | (4-1BB) | cells, B cells, DCs, |
| | | | endothelial cells |
| GITRL | Antigen-presenting cells | GITR | T and NK cells, Tregs |
| | and endothelium | | |
| ICOSLG | APCs, B cells, DCs and | ICOS | Naïve and activated T |
| | macrophages | | cells |
| CD70 | Activated lymphocytes | CD27 | Activated T and NK cells |

*taken from Marhelava et al. Cancers 2019, 11, 1756; doi: 10.3390/cancers11111756)]

Table 3 in the Examples section which follows outlines specific examples.

According to some embodiments, the immune checkpoint molecule is selected from the group consisting of CTLA4, PD-1, LAG3, TIGIT, TIM3, VISTA, CEACAM1, CD28, OX40, CD137(4-1BB), GITR, ICOS, CD27, CD80, CD86, PD-L1, PD-L2, MHC class II/lectins, CD155, Galectin 9, VSIG-3, B7, CD80, CD86, OX40L, CD137L, GITRL, ICOSLG and CD70.

For example, the development of a chimeric polypeptide for TIGIT will give a quantitative availability of all its ligands; CD112 and CD155, and for TIM3 will quantify the availability of Ceacam1, Gal-9, HMGB1 and PtdSer.

According to a specific embodiment, the immune checkpoint molecule is PD-1.

According to an exemplary embodiment the PD1 sequence is from NP_005009.2 (SEQ ID NO: 29).

According to a specific embodiment, the immune checkpoint molecule is CTLA-4.

According to an exemplary embodiment the CTLA-4 sequence is from NM_005214 (SEQ ID NO: 32).

According to a specific embodiment, the immune checkpoint molecule is LAG3.

According to an exemplary embodiment the LAG3 sequence is from X51985 (SEQ ID NO: 30).

According to a specific embodiment, the immune checkpoint molecule is TIM3.

According to an exemplary embodiment the TIM3 sequence is from AY069944 (SEQ ID NO: 31).

According to a specific embodiment, the immune checkpoint molecule is TIGIT.

According to an exemplary embodiment the TIGIT sequence is from NM 173799 (SEQ ID NO: 33).

Homologs of any of the contemplated sequences here are also included under the scope of the present invention according to some embodiments.

Thus, according to a specific embodiment, the amino acid sequence of the immune checkpoint molecule is a fragment or a homolog of the native immune checkpoint molecule, also referred to herein as functional equivalent, as long as it is capable of binding the ligand. According to a specific embodiment, it is devoid of the native transmembrane domain and cytoplasmic domain, which is replaced by that of the cell signaling module. According to a specific embodiment, the amino acid sequence of the immune checkpoint molecule comprises the extracellular domain which mediates ligand binding.

Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the native sequence, as long as the activity e.g., ligand binding is retained.

As used herein a “cell signaling module” refers to a portion of a signaling molecule that elicits signal transduction in a direct or indirect response to an extracellular signal.

“Activation” or “activated” in the context of signaling can be dimerization, protein-protein interaction, phosphorylation, de-phosphorylation, post-translational modification, migration, mobilization, combination of any of the foregoing or the like.

According to some embodiments, the portion is of a cell membrane receptor or cell membrane adapter associated with a signaling capacity that elicits signal transduction in a direct or indirect response to an extracellular signal.

Typically, the cell signaling module is of a cell surface receptor or associated with a cell-surface receptor e.g., T cell receptor complex, T cells co-stimulatory receptor, B-cell receptor complex, G protein—coupled receptor, cytokine receptors, growth factor receptor, tyrosine or Ser/Thr-specific receptor-protein kinase, integrin, Toll-like receptor, ligand gated ion channels or enzyme-linked receptors.

For example, the transmembrane and intracellular portion are of an enzyme-linked receptor. Various classes of enzyme-linked receptors are known and each of which is contemplated according to some embodiments of the invention. For example, receptor tyrosine kinase that phosphorylate specific tyrosines of intracellular signaling proteins; Tyrosine-kinase-associated receptors that associate with intracellular proteins that have tyrosine kinase activity; Receptor-like tyrosine phosphatases that remove phosphate groups from tyrosines of specific intracellular signaling proteins.; Receptor serine/threonine kinases that phosphorylate specific serines or threonines on associated latent gene regulatory proteins; Receptor guanylyl cyclases that directly catalyze the production of cyclic GMP in the cytosol; and Histidine-kinase-associated receptors activate a “two-component” signaling pathway in which the kinase phosphorylates itself on histidine and then immediately transfers the phosphate to a second intracellular signaling protein.

The binding of an extracellular signal (and in this case, ligand) typically changes the orientation of transmembranal structures, in some cases forming a dimer or a higher oligomer. In other cases the oligomirezation occurs before ligand binding and the ligand causes a reorientation of the receptor chains in the membrane. In either case, the rearrangement induced in cytoplasmic tails of the receptors initiates an intracellular signaling process.

Autophosphorylation of the cytoplasmic tail of receptor tyrosine kinases contributes to the activation process in two ways. First, phosphorylation of tyrosines within the kinase domain increases the kinase activity of the enzyme. Second, phosphorylation of tyrosines outside the kinase domain creates high-affinity docking sites for the binding of a number of intracellular signaling proteins in the target cell. Each type of signaling protein binds to a different phosphorylated site on the activated receptor because it contains a specific phosphotyrosine-binding domain that recognizes surrounding features of the polypeptide chain in addition to the phosphotyrosine. Once bound to the activated kinase, the signaling protein may itself become phosphorylated on tyrosines and thereby activated; alternatively, the binding alone may be sufficient to activate the docked signaling protein.

Alternatively, the signaling module is of a tyrosine phosphatase that acts as a cell surface receptor. Some comprise an SH2 domain and thus are called SHP-1 and SHP-2, additional compositions of signaling modules are described in the following references: SynNotch approach—cell 164, 1-10, Feb. 11, 2016 Protein-Logic based on HER2 and EGFR (M. J. Lajoie et al, Science 10.1126/science.aba6527 (2020), SUPRA-CAR technology (zipper TECHNOLOGY), Cell 173, May 31, 2018, incorporated herein by reference.

According to a specific embodiment, the cell signaling module is absent or inactive, or suppressed in the absence of stimulation or activation in the reporter cell.

According to a specific embodiment, the immune checkpoint molecule is naturally expressed on an immune cell and wherein the ligand is naturally expressed on a cancer cell.

According to a specific embodiment the immune checkpoint molecule is naturally expressed on a cancer cell and wherein the ligand is naturally expressed on an immune cell.

According to a specific embodiment the cell signaling module comprises a transmembrane domain and/or a cytoplasmic portion of a cell signaling receptor.

According to a specific embodiment the cell signaling module comprises a transmembrane domain and/or a cytoplasmic portion of a receptor kinase.

According to a specific embodiment the receptor kinase is a tyrosine kinase or serine/threonine kinase.

According to a specific embodiment the cell signaling module comprises an adaptor molecule.

According to a specific embodiment the cell signaling module comprises a CD3 zeta chain.

According to a specific embodiment the activation of the cell signaling module is by dimerization, oligomerization and/or post-translational modification.

According to a specific embodiment, the immune checkpoint (extracellular) is typically N-terminus to the cell signaling module (intracellular).

As used herein, the term “polypeptide” or “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells.

The term “amino acid” or “amino acids” typically refers to amino acids which can be used in recombinant protein synthesis.

When referring to “an amino acid sequence” the meaning is to the chemical embodiment of the term and not the literal embodiment of the term.

Alternatively or additionally, the polypeptides of some embodiments of the invention may be synthesized by any techniques that are known to those skilled in the art of peptide synthesis, such as, but not limited to, recombinant techniques.

Large scale peptide synthesis is described by Andersson Biopolymers 2000; 55 (3):227-50.

To express the chimeric polypeptide, the polynucleotide is cloned into a nucleic acid expression construct and introduced into a cell, i.e., a reporter cell.

Thus to express exogenous polynucleotides in cells, a polynucleotide sequence encoding the chimeric polypeptide is preferably ligated into a nucleic acid construct suitable for cell expression. Such a nucleic acid construct includes a promoter sequence for directing transcription of the polynucleotide sequence in the cell in a constitutive or inducible manner

As mentioned, the nucleic acid construct of some embodiments of the invention can also utilize nucleic acid homologues which exhibit the desired activity (e.g., ligand binding). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the native sequences, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with some embodiments of the invention are promoter sequences which are active under most environmental conditions and most types of cells such as the cytomegalovirus (CMV) and Rous sarcoma virus (RSV).

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for membrane presentation. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein.

Introduction of nucleic acids by viral infection offers several advantages over other methods such as lipofection and electroporation, since higher transfection efficiency can be obtained due to the infectious nature of viruses.

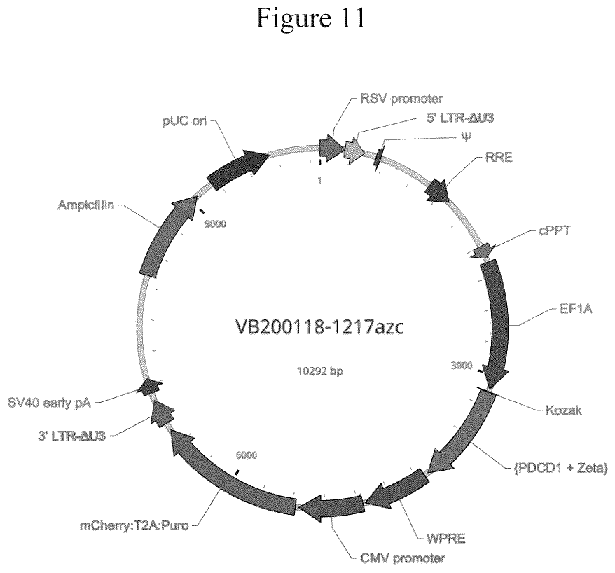

According to a specific embodiment, the vector is a Lentiviral vector e.g., as shown in FIG. 11.

Other than containing the necessary elements for the transcription and translation of the inserted coding sequence, the expression construct of some embodiments of the invention can also include sequences engineered to enhance stability, production, purification, yield or toxicity of the expressed peptide.

As mentioned hereinabove, a variety of prokaryotic or eukaryotic cells can be used as reporter-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence. Mammalian expression systems can also be used to express the polypeptides of some embodiments of the invention.

Thus, the reporter cell can also be referred to as a transgenic cell.

The polynucleotide of some embodiments of the invention can be introduced into cells by any one of a variety of known methods within the art. Such methods can be found generally described in Sambrook et al., [Molecular Cloning: A Laboratory Manual, Cold Springs Harbor Laboratory, New York (1989, 1992)]; Ausubel et al., [Current Protocols in Molecular Biology, John Wiley and Sons, Baltimore, Maryland (1989)]; Chang et al., [Somatic Gene Therapy, CRC Press, Ann Arbor, MI (1995)]; Vega et al., [Gene Targeting, CRC Press, Ann Arbor MI (1995)]; Vectors [A Survey of Molecular Cloning Vectors and Their Uses, Butterworths, Boston MA (1988)] and Gilboa et al. [Biotechniques 4 (6): 504-512 (1986)] and include, for example, stable or transient transfection, lipofection, electroporation and infection with recombinant viral vectors. Introduction of the polynucleotide can be in a stable or transient manner

The “reporter cell” is any cell which can be used as a host cell for recombinant expression of the polynucleotide and in which the cell signaling module is capable of eliciting signaling.

According to some embodiments, the reporter cell can be a cell line or a primary cell.

According to some embodiments, the reporter cell is typically isolated and does not form a part of a tissue.

According to some embodiments, the reporter cell is an immune cell, e.g., T lymphocyte, B lymphocyte and the like.

According to a specific embodiment, the cell is a mammalian cell, e.g., human or murine cell.

According to a specific embodiment, the immune cell is an antigen presenting cell.

According to a specific embodiment, the immune cell is not an antigen presenting cell.

According to some embodiments, the reporter cell is a non-immune cell which is typically used for recombinant expression, e.g., CHO, 293T, NIII3T3, COS7 and the like.

The reporter cell can express more than one polynucleotide to decipher expression or activity of a plurality of ligands e.g., PD-1 and CTLA-4, in such a case the signaling module may be different for each immune checkpoint molecule or a single signaling module may be used but the ligands are added sequentially for instance.

Activation of the signaling module can be done by detecting induction (e.g., expression) of a reporting molecule (e.g., IL-2, IL-8) or a fluorescent or bioluminescent signal, for instance using an promoter responsive element(s), responding at the end of the signaling module cascade, linked to anucleic acid sequence encoding a bioluminescent or fluorescent molecule.

According to a specific embodiment, the reporter gene encodes an enzyme whose catalytic activity can be detected by a simple assay method or a protein with a property such as intrinsic fluorescence or luminescence so that expression of the reporter gene can be detected in a simple and rapid assay requiring minimal sample preparation. Non-limiting examples of enzymes whose catalytic activity can be detected are Luciferase, beta Galactosidase, Alkaline Phosphatase.

The term “protein with intrinsic fluorescence” refers to a protein capable of forming a highly fluorescent, intrinsic chromophore either through the cyclization and oxidation of internal amino acids within the protein or via the enzymatic addition of a fluorescent co-factor. The term “protein with intrinsic fluorescence” includes wild-type fluorescent proteins and mutants that exhibit altered spectral or physical properties. The term does not include proteins that exhibit weak fluorescence by virtue only of the fluorescence contribution of non-modified tyrosine, tryptophan, histidine and phenylalanine groups within the protein. Proteins with intrinsic fluorescence are known in the art, e.g., green fluorescent protein (GFP),), red fluorescent protein (RFP), Blue fluorescent protein (BFP, Heim et al. 1994, 1996), a cyan fluorescent variant known as CFP (Heim et al. 1996; Tsien 1998); a yellow fluorescent variant known as YFP (Ormo et al. 1996; Wachter et al. 1998); a violet-excitable green fluorescent variant known as Sapphire (Tsien 1998; Zapata-Hommer et al. 2003); and a cyan-excitable green fluorescing variant known as enhanced green fluorescent protein or EGFP (Yang et al. 1996) and can be measured e.g., by live cell imaging (e.g., Incucyte) or fluorescent spectrophotometry. “Reduced binding” refers to a decrease in affinity for the respective interaction, as measured for example by SPR. For clarity the term includes also reduction of the affinity to zero (or below the detection limit of the analytic method), i.e. complete abolishment of the interaction.

The method can use more than one reporter e.g., a first reporter and a second reporter, which are different in the signal they produce. The second reporter can be used to detect an organelle for instance, such as to mark a cell membrane, a cell nucleus, a cell cytoplasm and the like. The second reporter can be also a chemical dye i.e., non-proteinaceous.

According to a specific embodiment, the first reporter and optionally second reporter are fluorescent or bioluminescent.

Alternatively or additionally, determining activation is by analyzing a phenotype selected from the group consisting of cell proliferation, death, arrest, migration, morphology, cell localization in a tissue, receptor ligand interactions and the like.

Methods of analyzing interleukin in culture are well known in the art and some are based on commercially available kits.

It will be appreciated that due to the high sensitivity of the cells, the methods described herein can be employed using as little as 10² cells or at least 10² cells (e.g., 10²-10⁴, 10²-10³, 10²-5×10³).

The reporter cells described herein can be used in methods which qualify/quantify immune checkpoint ligands on cancer cells.

Thus, according to an aspect of the invention, there is provided a method of detecting presence and/or activity of a ligand of an immune checkpoint molecule in a cancer cell, the method comprising:
(a) contacting the cancer cell or a cell in a microenvironment of the cancer cell with the reporter cell as described herein; and
(b) determining activation of the cell signaling module in the reporter cell, the activation being indicative of the presence and/or activity of the ligand of the immune checkpoint molecule in the cancer cell.

According to another aspect of the invention, there is provided a method of detecting presence and/or activity of a receptor of an immune checkpoint molecule in an immune cell, the method comprising:
(a) contacting the immune cell with the reporter cell comprising a polynucleotide encoding a chimeric polypeptide comprising an amino acid sequence of an immune checkpoint molecule capable of binding a receptor thereof, the immune checkpoint molecule being translationally fused to another amino acid sequence of a cell signaling module such that upon binding of the immune checkpoint molecule to the receptor, the cell signaling module is activated;
(b) determining activation of the cell signaling module in the reporter cell, the activation being indicative of the presence and/or activity of the receptor of the immune checkpoint molecule in the immune cell.

As used herein, the term “cancer” encompasses both malignant and pre-malignant cancers.

Cancers which can be analyzed and eventually treated by the methods of some embodiments of the invention can be any solid or non-solid cancer and/or cancer metastasis. According to a specific embodiment, the cancer is a solid tumor.

Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; Burkitt lymphoma, Diffused large B cell lymphoma (DLBCL), high grade lymphoblastic NHL; high-grade small non-cleaved cell NHL; bulky disease NHL; mantle cell lymphoma; AIDS-related lymphoma; and Waldenstrom's Macroglobulinemia); T cell lymphoma, Hodgkin lymphoma, chronic lymphocytic leukemia (CLL); acute lymphoblastic leukemia (ALL); Acute myeloid leukemia (AML), Acute promyelocytic leukemia (APL), Hairy cell leukemia; chronic myeloblastic leukemia (CML); and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention also include metastatic cancers.

According to specific embodiments, the cancer comprises pre-malignant cancer.

Pre-malignant cancers (or pre-cancers) are well characterized and known in the art (refer, for example, to Berman J J. and Henson D E., 2003. Classifying the precancers: a metadata approach. BMC Med Inform Decis Mak. 3:8). Classes of pre-malignant cancers amenable to treatment via the method of the invention include acquired small or microscopic pre-malignant cancers, acquired large lesions with nuclear atypia, precursor lesions occurring with inherited hyperplastic syndromes that progress to cancer, and acquired diffuse hyperplasias and diffuse metaplasias.

Examples of small or microscopic pre-malignant cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is Acute Lymphocytic Leukemia (ALL), Acute Myeloid Leukemia, Anal Cancer, Basal Cell Carcinoma, B-Cell Non-Hodgkin Lymphoma, Bile Duct Cancer, Bladder Cancer, Breast Cancer, Cervical Cancer, Chronic Lymphocytic Leukemia (CLL), Chronic Myelocytic Leukemia (CML), Colorectal Cancer, Cutaneous T-Cell

Lymphoma, Diffuse Large B-Cell Lymphoma, Endometrial Cancer, Esophageal Cancer, Fallopian Tube Cancer, Follicular Lymphoma, Gastric Cancer, Gastroesophageal (GE) Junction Carcinomas, Germ Cell Tumors, Germinomatous (Seminomatous), Germ Cell Tumors, Glioblastoma Multiforme (GBM), Gliosarcoma, Head And Neck Cancer, Hepatocellular Carcinoma, Hodgkin Lymphoma, Hypopharyngeal Cancer, Laryngeal Cancer, Leiomyosarcoma, Mantle Cell Lymphoma, Melanoma, Merkel Cell Carcinoma, Multiple Myeloma, Neuroendocrine Tumors, Non-Hodgkin Lymphoma, Non-Small Cell Lung Cancer, Oral Cavity (Mouth) Cancer, Oropharyngeal Cancer, Osteosarcoma, Ovarian Cancer, Pancreatic Cancer, Peripheral Nerve Sheath Tumor (Neurofibrosarcoma), Peripheral T-Cell Lymphomas (PTCL), Peritoneal Cancer, Prostate Cancer, Renal Cell Carcinoma, Salivary Gland Cancer, Skin Cancer, Small-Cell Lung Cancer, Soft Tissue Sarcoma, Squamous Cell Carcinoma, Synovial Sarcoma, Testicular Cancer, Thymic Carcinoma, Thyroid Cancer, Ureter Cancer, Urethral Cancer, Uterine Cancer, Vaginal Cancer or Vulvar Cancer.

According to specific embodiments, the cancer is Acute myeloid leukemia, Bladder Cancer, Breast Cancer, chronic lymphocytic leukemia, Chronic myelogenous leukemia, Colorectal cancer, Diffuse large B-cell lymphoma, Epithelial Ovarian Cancer, Epithelial Tumor, Fallopian Tube Cancer, Follicular Lymphoma, Glioblastoma multiform, Hepatocellular carcinoma, Head and Neck Cancer, Leukemia, Lymphoma, Mantle Cell Lymphoma, Melanoma, Mesothelioma, Multiple Myeloma, Nasopharyngeal Cancer, Non Hodgkin lymphoma, Non-small-cell lung carcinoma, Ovarian Cancer, Prostate Cancer or Renal cell carcinoma.

According to specific embodiments, the cancer is selected from the group consisting of Acute Lymphocytic Leukemia (ALL), Bladder Cancer, Breast Cancer, Colorectal Cancer, Head and Neck Cancer, Hepatocellular Carcinoma, Melanoma, Multiple Myeloma, Non-Small Cell Lung Cancer, Non-Hodgkin Lymphoma, Ovarian Cancer, Renal Cell Carcinoma.

According to specific embodiments, the cancer is selected from the group consisting of Gastrointestinal (GI) cancers, Breast Cancer, Ovarian Cancer and Pancreatic Cancer.

The cancer cell can be a primary cell taken from a tissue biopsy or a cell line.

According to a specific embodiment, the cancer cell is comprised in a tissue biopsy.

According to some embodiments, the tissue biopsy is fresh, not subjected to any preservation protocol. i.e., fixation protocol.

According to other embodiments, the tissue biopsy has been subject to fixation.

According to some embodiments, the tissue biopsy is subjected to antigen retrieval.

For example, when the tissue biopsy has been preserved with formaldehyde, a highly reactive compound, it may a variety of chemical modifications that can reduce the detectability of proteins in biomedical procedures. Antigen retrieval is an approach to reducing or eliminating these chemical modifications. The two primary methods of antigen retrieval are heat-mediated epitope retrieval (HIER) and proteolytic induced epitope retrieval (PIER).

Thus, contacting with the reporter cell is preferably and according to some embodiments of the invention done following antigen retrieval.

The cancer cell can be used following freezing/thawing or immediately upon biopsy retrieval.

According to a specific embodiment, the cancer cell is a primary cell.

Contacting can be effected in a culture dish such as in a petri dish or flask, or in a multiwall configuration e.g., 96 or more wells, when a plurality of ligands are assayed and/or a plurality of immune checkpoint modulators.

According to some embodiment, the contacting is effected such that the tumor tissue is seeded on the plate and the reporter cells are seeded thereon.

Contacting can be effected in the presence and/or absence of an immune checkpoint modulator or a plurality of immune checkpoint modulators.

As used herein “an immune checkpoint modulator” refers to an agent that modulates the immune checkpoint pathway, either by blocking any inhibitory immune checkpoint protein or by activating any stimulatory immune checkpoint protein.

According to some embodiments of the invention, the immune checkpoint modulator is an antibody.

Following is a non-limiting list of modulators which can be used in accordance with some embodiments of the invention.

TABLE 2
The list of Food and Drug Administration (FDA)-approved monoclonal antibodies
acting as inhibitors of negative checkpoints in human cancer.*

| | | Examples of Types of Cancers | Year of First |
|---|---|---|---|
| Checkpoint Inhibitor | Antibody Format | with FDA-Approved Use | Approval |
| Ipilimumab | Human anti-CTLA4 | Melanoma, renal cell carcinoma, | 2011 |
| | IgG1 | metastatic colorectal cancer | |
| Pembrolizumab | Humanized | Melanoma, non-small-cell lung | 2014 |
| | anti-PD-1 IgG4 | cancer, renal cell carcinoma, | |
| | | urothelial bladder cancer, Hodgkin | |
| | | lymphoma, head and neck cancer, | |
| | | Merkel cell carcinoma, microsatellite | |
| | | instability-high cancer, gastric cancer, | |
| | | hepatocellular carcinoma, cervical | |
| | | cancer, primary mediastinal large | |
| | | B-cell lymphoma | |
| Nivolumab | Human anti-PD-1 | Melanoma, non-small-cell lung | 2014 |
| | IgG4 | cancer, renal cell carcinoma, | |
| | | urothelial bladder cancer, Hodgkin | |
| | | lymphoma, head and neck cancer, | |
| | | colorectal cancer, hepatocellular | |
| | | carcinoma, small cell lung cancer | |
| Atezolizumab | Humanized | Non-small-cell lung cancer, urothelial | 2016 |
| | anti-PD-L1 IgG1 | bladder cancer, small cell lung cancer, | |
| | | breast cancer | |
| Avelumab | Human anti-PD-L1 | Merkel cell carcinoma, urothelial | 2017 |
| | IgG1 | bladder cancer | |
| Durvalumab | Human anti-PD-L1 | Non-small-cell lung cancer, urothelial | 2017 |
| | IgG1 | bladder cancer | |
| Cemiplimab | Human anti-PD-1 | Cutaneous squamous-cell carcinoma | 2018 |
| | IgG4 | | |

*taken from Marhelava et al. Cancers 2019, 11, 1756; doi: 10.3390/cancers11111756)]

According to a specific embodiment, the immune checkpoint modulator is an anti-PD-1.

Contacting can be effected first, between the cancer cell and the reporter cell and then subjecting to the immune modulator.

Alternatively, contacting can be effected in the presence of the immune checkpoint modulator (simultaneous incubation). Other configurations are also contemplated. For example, contacting can be effected in the presence of a soluble ligand (e.g. soluble PDL-1).

Activation of the cell signaling module is determined using methods known in the art and available kits.

Typically, activation is determined relative to a control, such as a negative control to determine base activation of the cell signaling module.

According to some embodiments, the negative control is under the same conditions yet in the absence of the immune checkpoint modulator or with isotype matched control. Alternatively using normal cells which are adjacent to the tumor (e.g., on the same tissue sample, see for instance Example 9). Such a control can also be used to determine treatment toxicity to normal tissues.

Activation of the cell signaling module is indicative of the presence and/or activity of the ligand of the immune checkpoint molecule in the cancer cell.

It will be appreciated that effect of the modulator on the activation is indicative of the specificity of activation of the cell signaling module.

It will be further appreciated that the results of the assay can be corroborated by testing immune cells of the subject with a chimeric polypeptide in which the ligand of the immune checkpoint molecule is expressed in the reporter cell.

The level of activation can be calculated using various algorithms including those which employ scoring. In such a case, the scoring of the response may be based on a scoring combination of (a) the level of activation without the immune modulator (i.e., maximal activation of the reporter cell); and (b) the fraction of reduction of activation after adding the immune modulator.

According to some embodiments, the quantification of the ligand (or immune checkpoint molecule) is done without immunohistochemistry (IHC).

According to some embodiments, the quantification of the ligand (or immune checkpoint molecule) is corroborated by immunohistochemistry (111C).

According to some embodiments, the quantification of the ligand (or immune checkpoint molecule) is corroborated by transcriptome analysis.

These teachings can be harnessed towards selecting treatments for cancer patients.

Thus, according to an aspect of the invention there is provided a method of treating a subject diagnosed with cancer, the method comprising:
(a) contacting the cancer cell or a cell in a microenvironment of the cancer cell of the subject with the reporter cell of as described herein;
(b) determining activation of the cell signaling module in the reporter cell, the activation being indicative of the presence and/or activity of the ligand of the immune checkpoint molecule in the cancer cell; and
(c) treating the subject with a modulator of the immune checkpoint molecule when presence or a predetermined threshold of activity of the ligand of the immune checkpoint molecule is indicated or with another treatment modality when it is not indicated or absent.

As used herein “a cell of a microenvironment of the cancer cell” refers to the tumor microenvironment (TME) which is a non-cancer TME such as macrophage/dendritic cell that can also express the ligand.

As used herein “predetermined threshold” typically refers to at least above 20%, 30%, 40%, 50%, 70%, 2 fold, 5 fold 10 fold or more increase in activity as compared to a negative control in a statistically significant manner

It will be appreciated that a scoring system can be employed to elucidate activation above a “predetermined threshold”. Such a scoring system can take into account the difference in activation between the presence and absence of the the immune modulator. Additionally the surface of each well covered by the patients-derived tissue is taken into account. Calculation of the covered area (tissue surface) is made by imaging analysis of each individual well.

According to a specific embodiment the scoring system is an IcAR-score, based on:

Calculation of IcAR score—The IcAR score is based on calculation between the maximum signal (PC), and the signal obtained with and without blocking with the immune modulator. Moreover, also taken into account was the area of the tissue (surface) that covers the 96 well plates. To compare between experiments and plates the present inventors have used the PC. PCavg—is pooled of all experiments, PCexp—is the positive control of the specific experiment.

$$IcAR score = \{ ( \frac{AvgIL 2 unblocked}{Log 2 surface} ) - ( \frac{AvgIL 2 blocked}{Log 2 surface} ) \} * \frac{PCAvgIL 2}{PC exp IL 2}$$

According to an alternative or an additional aspect there is provided a method of selecting treatment for a subject diagnosed with cancer, the method comprising:
(a) contacting the cancer cell or a cell in a microenvironment of the cancer cell of the subject with the reporter cell of as described herein;
(b) determining activation of the cell signaling module in the reporter cell, the activation being indicative of the presence and/or activity of the ligand of the immune checkpoint molecule in the cancer cell; and
(c) selecting treatment for the subject with a modulator of the immune checkpoint molecule when presence or a predetermined threshold of activity of the ligand of the immune checkpoint molecule is indicated or with another treatment modality when it is not indicated or absent.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

According to a specific embodiment, the cancer cell is autologous to the subject.

According to a specific embodiment, the immune cell is autologous to the subject.

It is expected that during the life of a patent maturing from this application many relevant immune checkpoint modulators will be developed and the scope of the term is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Tissue Sections

The FFPE 4-5 micron sections are attached on negatively charged coverslip. The tissue sections were de-deparaffinized with xylene (3 times, 10 minutes each). Then washed well with PBS and fixation with Ethanol. Wash again three times. For Antigen retrieval, 10 mM of acetic acid pH=6 and heated in a boiling water bath (99° C.) for 30 minutes. Wash well with PBS.

Transfer the coverslips into tissue culture plate (24 well plate) and seed 1 million AR cells on top of the coverslip for 18 hours. Control is assessed by stimulating Ab, negative control, no tissue, and on target effect assessed by neutralization Ab.

Supernatant is collected 18 hours later, and ELISA for IL-2 is assessed.

### Cloning

A lenti-viral vector was designed to overexpress the chimeric proteins. The vector includes selection of puro, and cloning sites. See FIG. 11. The specific sequences are shown is SEQ ID Nos: 16-28.

### Cell Lines

Mouse BW5147 thymoma cells (ATCC TIB-47™) and transfectants were maintained in RPMI 1640 medium containing 10% (v/v) FCS, penicillin, streptomycin, glutamine, and sodium pyruvate (1 mM). BW-derived reporter cell lines were maintained in RPMI medium containing selection antibiotics.

Human lung carcinoma cell line A549 (ATCC CCL-185™) were maintained in DMEM medium containing 10% (v/v) FCS, penicillin, streptomycin, glutamine, and sodium pyruvate (1 mM).

### Cell Co-culture

The A549 cells were seeded in flat 96-well plates at a density of 0.25×10⁵ cells/well and cultured in the absence or presence of IFN-γ for 24 h. The A549 cells were then co-cultured with 100,000 (at density of 1×10⁶ cell/ml) Artificial reporter cells for 20-24 h. Supernatant was collected 20-24 hours later followed by testing with a murine IL-2 commercial sandwich ELISA kit (Biolegend, CA USA).

### Cloning Fusion Protein Immunododulators and Zeta Chain

Cloned sequences encoding the human extracellular regions of several immune-checkpoint receptors fused to murine CD3ζ chain were ordered from (HyLabs, Israel) immune-checkpoint receptors included PD-1, CTLA-4, TIGIT, LAG3 and TIM3. Extracellular portions of PD-1 cDNA (NM_005018.2; (24F-170V)), CTLA-4 cDNA (NM_001037631.2; (36K-161D), TIGIT cDNA (NM_173799.3; (22M-141P)), LAG3 cDNA (NM_002286.5; (23L-450L)), and TIM3 cDNA (NM_032782.5; (22s-202G) were fused to mouse CD3ζ chain cDNA (NM_001113391.2; (31L-164R)) making PD-1-ζ, CTLA4-ζ, TIGIT-ζ, LAG3-ζ and TIM3-ζ respectively. The immune-checkpoint receptors sequences were cloned into pHAGE240.

### Viral Production and Preparation of Reporter Cells

HEK-293T cells were transiently transfected with plasmids of interest and lentiviral packaging plasmids, and retrovirus (RV)-containing supernatants were harvested, aliquoted and employed for transduction of BW5147 thymoma cells. Following selection with Puromycin (Invivogen, CA USA) /G418 (Sigma-Aldrich, Israel), stable transfectants were screened by flow cytometry using antibodies for anti-PD1(#621609 BioLegend, CA USA), anti-CTLA4 (#349902 BioLegend, CA USA), anti-TIGIT (#372702 BioLegend, CA USA), anti-TIM3 (#MABF62 Sigma-Aldrich, MO USA) and anti-LAG3 (#369302 BioLegend, CA USA). Cells were then stained using secondary goat anti-mouse IgG APC (Jackson immunoresearch, PA USA). BW5147 expressing receptor fused to murine CD3ζ secrete IL-2 following activation of the receptor.

### In Vitro IcAR-activation

To further test function of transfectants, IcAR-cells were incubated with A549 cells. To upregulate PD-L1 and PD-L2 expression A549 were pre-incubated with interferon-gamma (IFN-g) or control vehicle—PBS. Followed such co-caltutre the murine IL-2 was tested by a commercial sandwich ELISA kit (Biolegend, CA USA).

### FFPE Co-culture

FFPE tissue was prepared according to standard procedures. The 5 micron tissue sections were de-deparaffinized with xylene (3 times, 10 minutes each). Samples were then hydrated gradually through graded alcohols: wash in 100% ethanol (twice, 5 minutes each), 95% ethanol (once, 5 minutes) and 70% ethanol (once, 5 minutes). Samples were then washed in deionized H₂O (3 times, 5 minutes each). For Antigen retrieval, samples were placed in 10 ml of Tris-EDTA (10 mM acid pH=9) and heated in a boiling water bath (95° C.) for 30 minutes. Samples were then washed in deionized H2O (3 times, 5 minutes each).Then, the FFPE 5-micron sections were attached to 96 well plate.

For co-culture, 500,000 (at density of 2×10⁶ cell/ml) AR cells were seeded on top of the tissue for 20-24 h hours. Control was assessed by stimulating Ab (Pembrolizumab), negative control (no tissue) and on target effect assessed by neutralization AB (Durvalumab). The supernatant was collected 20-24 hours later, followed by testing with a murine IL-2 commercial sandwich ELISA kit (Biolegend, CA USA). For blocking the signal and for calculating the IcAR-score antibodies that block the receptor or ligands have been used. Specifically, used was 10 ug/ml of Keytruda or Nivolumab

### Antibodies:

For coating the plates with Ab, and for blocking the receptors/ligands for IcAR-Pd1 the present inventors have used the clinically approved Abs: Keytroda, Durvalumab and Nivolomab 10 ug/ml. For other IcAR the following were used: BioLegend #369302—for LAG3, Sigma-Aldrich #MABF62 for TIM3, BioLegend #349902 for CTLA4, BioLegend #372702—for TIGIT.

### IcAR-score

Calculation of IcAR score—The IcAR score is based on calculation between the maximum signal (PC), and the signal obtained with and with drug that inhibit the receptor/ligand interaction. Moreover, also taken into account the area of the tissue (surface) that covers the 96 well plates. To compare between experiments and plates the present investigators have used the PC. PCavg—is pooled of all experiments, PCexp—is the positive control of the specific experiment.

$$IcAR score = \{ ( \frac{AvgIL 2 unblocked}{Log 2 surface} ) - ( \frac{AvgIL 2 blocked}{Log 2 surface} ) \} * \frac{PCAvgIL 2}{PC exp IL 2}$$

Evaluation of IcAR Activation by mIL-2 Measurement

The supernatant of activated cells (as described above) was collected after 20-24 h incubation and analyzed for mIL-2 by ELISA assay. 96 well ELISA plates were pre-coated with purified anti-mouse IL-2 (Biolegend, CA USA) using coating buffer (0.1 M, Na2HPO; pH 9.0), blocked with 10% FBS in PBST (0.05% Tween-20), coated with collected supernatant followed by addition of biotinylated anti-mouse IL-2 (Biolegend, CA USA), and then SA-HRP (Jackson immunoresearch, PA USA) and TMB (Dako, Denmark) for detection of mIL-2

### Statistical Analysis

Statistical analyses were performed with GraphPad Prism8. Data in bar graphs are presented as means±SD/SEM. The association of IL-2 secretion and PD-L1 expression was analyzed using two-way ANOVA. Differences were considered to be statistically significant at a two-side * for p<0.05, and ** for p<0.001.

### EXAMPLE 1

### Generation of IcAR for PD-1 (IcAR-PD-1)

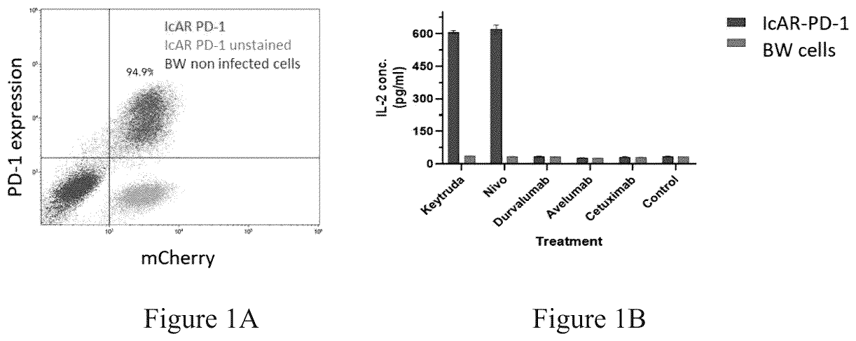

Using protein expression databases, the present inventors built a vector that combines the extracellular domains of the PDDC1 receptor with the transmembrane and intracellular domains of the CD-3 zeta chain. A stable cell line, BW5147, was transformed with the vector to overexpress this fusion protein named as IcAR-PD-1, (FIG. 1A). To test the ability of IcAR-PD1 to transmit a signal after stimulation or after recognizing PD-L1, the binding and signal was tested in the presence of selected anti-PD-1 therapies. As anti-PD-1 monoclonal antibodies, Keytruda and Nivolumab, were used. These antibodies, when coated on a firm surface and exposed to the PD-1 artificial reporter, bind to the extracellular portion of IcAR-PD-1 and activate the signaling pathway to secrete IL-2 detected by ELISA (FIG. 1B).

### EXAMPLE 2

### IcAR-PD-1 is a Sensitive Tool for Recognizing PD-L1 Expression

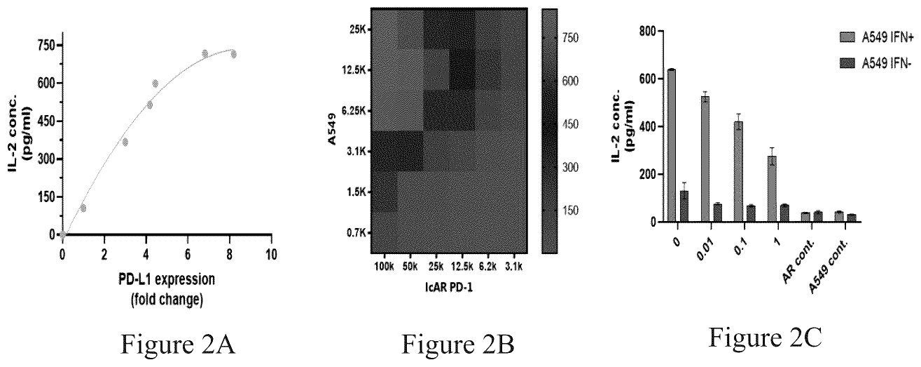

To test IcAR-PD-1 response to PD-L1 expression in live cells, the present inventors used human adeno-carcinomic alveolar cell line—A549 and upregulated PD-L1 expression using recombinant IFN gamma (rIFN [symbol] ). The results shown in FIG. 2A, indicate a correlation between PD-L1 expression (measured by FACS) and IcAR-PD-1 stimulation measured by IL-2 secretion. To quantify the minimal amount of tissue required for detection, the present inventors defined the optimal conditions by measuring IL-2 secretion after co-culture of A549 cells with IcAR-PD-1 in different ratios (FIG. 2B). These calibration experiments indicate that 3000 cells that express PD-L1 are sufficient to provide a strong signal. This low number of cells become crucial when limited tissue is available. Notably, it was possible to prevent the IcAR-PD-1 activation by blocking PD-L1 expression in tumor cells using Durvalumab and Avelumab (FIG. 2C).

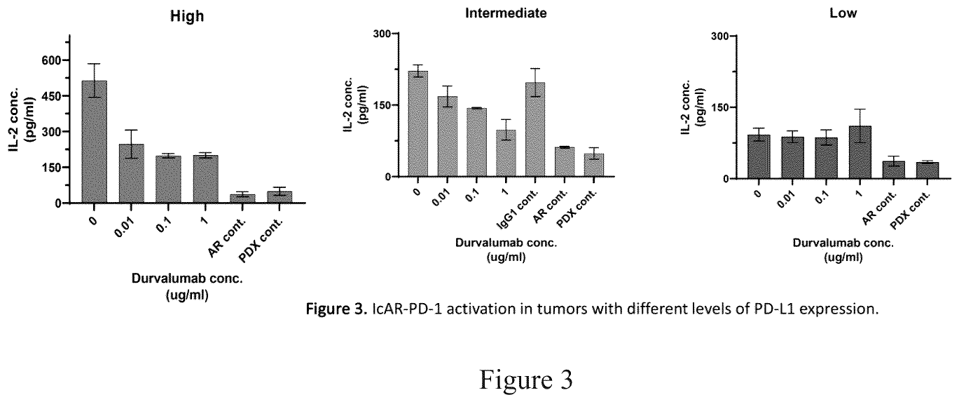

To further examine IcAR-PD-1 capability to recognize PD-L1, the present inventors used both fresh PDX and human tumor samples obtained by tumor section. Specifically, IL-2 secretion was quantified 18 hours following co-culture of tumor tissue with IcAR-PD-1 and the specificity of the IcAR activation was verified by blocking PD-L1 using Durvalumab and Avelumab (FIG. 3).

### EXAMPLE 3

### Designed IcARs for CTLA4, TIM3, TIGIT, BTLA, VISTA, CD96, MHC-class I and LAG3

The present inventors defined the protein sequences from genomic databases of immunomodulators CTLA4 TIM3, TIGIT, BTLA, VISTA, CD96, MHC-class I and LAG3 as shown in Table 3). Moreover, the ligands, Abs to measure IcAR activity with their product number, are also listed in Table 3.

TABLE 3
Detailed information on IcARs according to some embodiments of the invention

| | Gene name | | Length | AB | |
|---|---|---|---|---|---|
| | and Entrez | | (Amino | (Functional | Blocking |
| Protein | Gene | Sequence | acids) | assay) | assay |
| PD-1 | PDCD1 | MQIPQ . . . FQTLV | 170 | Pembrolizumab | Durvalumab, |
| | 5133, e.g., | | | | Avelumab |
| | NP_005009.2 | | | | |
| LAG3 | LAG3 | MWEA . . . PAGHL | 450 | 369302 | Relatlimab |
| | 3902, e.g., | | | | |
| | X51985 | | | | |
| TIM3 | HAVCR2 | MFSHL . . . TIRIG | 202 | Sigma- | rhGal-9 |
| | 84868 e.g., | | | Aldrich #MABF62 | Bio- |
| | AY069944 | | | | Legend #557302 |
| CTLA4 | CTLA4 | MACLG . . . CPDSD | 161 | 349902 | Ipilimumab |
| | 1493 | | | BioLegend # | |
| | e.g., | | | | |
| | NM_005214 | | | | |
| TIGIT | TIGIT | MRWCL . . . RFQIP | 141 | Bio- | Anti-CD155 |
| | 201633 | | | Legend #372702 | Thermo- |
| | e.g., | | | | fisher #46155042 |
| | NM_173799 | | | | |
| BTLA | BTLA | MKTLP . . . WLLYR | 157 | Thermo- | Anti-CD270 |
| | 151888 | | | fisher #14597982 | Bio- |
| | AY293286 | | | | legend #318802 |
| CD96 | CD96 | MEKKW . . . PKDGM | 519 | 338402 | Anti-CD155 |
| | 10225 | | | BioLegend # | Thermo- |
| | NM_005816.4 | | | | fisher #46155042 |
| | or | | | | |
| | NM_198196.2 | | | | |
| Anti MHC-1 | W6-32 | MKSQT . . . TRGLD | 339 | A549, HeLa, | W6/32 |
| Recognizes | | | | Cal33 cell lines | Bio- |
| HLA | | | | | legend #311402 |
| A, B, C | | | | | |

### EXAMPLE 4

### IcAR-PD-1 Recognizes PD-L1 Expression on FFPE Samples

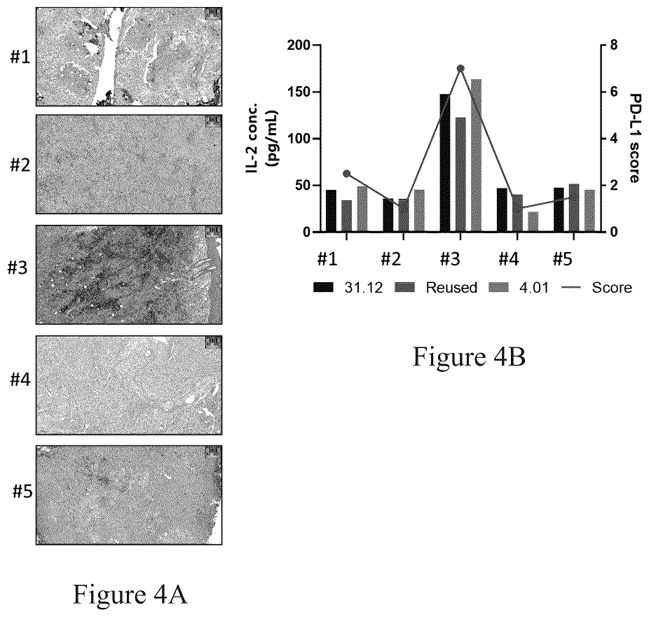

After validating IcAR PD-1 ability to recognize and respond to PD-L1 on fresh tissue samples (FIGS. 4A-B), the present inventors aimed to develop IcAR-PD-1 to recognize PD-L1 on FFPE tissues. To this end, the present inventors developed a well-defined protocol for antigen retrieval that enables the specific binding between PD-L1 on the tissue and PD-1 on the IcAR. Specifically, 5 micron of tissue was attached to negatively charged slide/coverslip and the slides/coverslips were inserted into tissue culture-type wells, like 6- or 24-well plates. Paraffin blocks of head and neck tumors with a known PD-L1 staining score were used. The IL-2 levels and PD-L1 staining shows a similar trend, indicating the accuracy of the IcAR system to measure ligand activity. To further examine IcAR-PD-1 capability to recognize PD-L1 in FFPE samples, the present inventors used the PDX cohort to compare side by side fresh and fixated samples. FIGS. 4A-B show the correlation of IcRA-PD1 activation in both approaches, which further confidence that IcAR works reliably in FFPE.

### EXAMPLE 5

### IcAR-PD1 Recognizes PD-L1 Equally in Fresh and FFPE Samples, and FFPE can be Re-used for Multiple IcARs

Because tumor samples, mainly biopsies, are limited and in many cases, only a few sections are available, the present inventors first tested if fresh and FFPE sample give a similar trend on IcAR-PD-1 activation. FIGS. 4A-B show the correlation between IcAR-PD-1 activation on matched FFPE and fresh samples. The present inventors next explored if the FFPE samples can be re-used. To test that, FFPE samples used in FIGS. 4A-B were taken and treated to remove all the IcAR-PD-1 by washing with PBS before adding fresh IcAR-PD1 cells on the tissue. IL-2 levels show that the new IcAR-PD-1 cells get stimulated to the same degree as for the first test. These results point to the possibility that it is possible to test several IcARs on a single slide.

### EXAMPLE 6

### IcAR Assay Accurately Predicts the Clinical Response to PD1 Therapy as Shown in Lung Cancer Lesions

The IcAR assay was implemented on cuts taken from FFPE blocks of human lung cancer lesions. Cancers were either NSCLC or from other etiologies that resulted in the subsequent growth of lung cancer lesions, from which FFPE samples were generated. Following the generation of the FFPE sample, patients began immunotherapy with anti PD1.

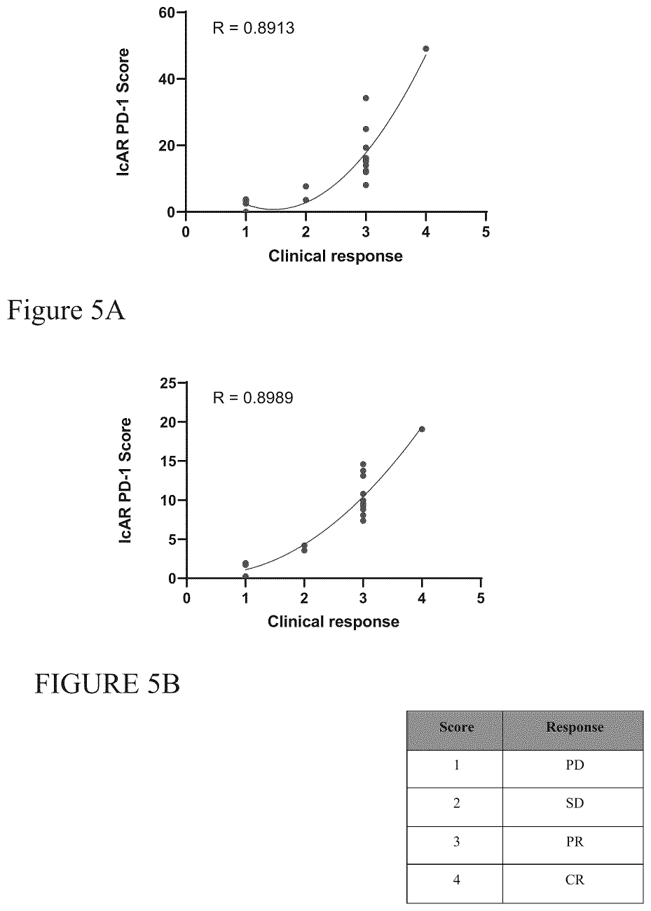

The IcAR assay was performed using the the artificial reporter IcAR-PD1 of Example 1 above and employed in the assay either anti PD1 drug (pembrolizumab) or anti PDL1 drug (durvalumab); As shown in FIGS. 5A-B, although the IcAR assay scores with pembrolizumab were approximately 2-fold higher than the assay scores with durvalomab, the overall ratios among the scores of the various samples remained similar

Thereafter, the correlation of IcAR-PD1 scores were assessed for correlation with the clinical results. FIGS. 5A-B show the association of IcAR-PD1 score, determined either with pembrolizumab (FIGS. 5A-B, left panel) or with durvalumab (FIGS. 5A-B, right panel) employed in the IcAR assay. Association (direct correlation) between IcAR-PD1 score (either when using pembrolizumab or durvalumab in the assay) and the clinical response (values: CR=1, PR=2, SD=3, and PD=4) was very high and statistically significant (Spearman R=0.8989, p<0.0001 when employing pembrolizumab in the assay, and Spearman R=0.8913, p<0.0001 when employing durvalumab in the assay).

### EXAMPLE 7

### IcAR Assay Performed in Some Embodiments of the Invention with an Antibody to the Receptor and an Antibody to the Ligand Due to the Existence of Different Ligands to a Single IC Receptor

Note that in Example 6, when the ICAR-PD1 score was compared to the clinical results, the samples were assayed with either an anti-PD1 or an anti PDL1. This is because the PD1 receptor has two known ligands (PDL1 and PDL2). When one treats the cancer patient with an IC blocker based on an antibody to the receptor (e.g. anti PD1) then the function of effector cells suppressed through PD1 receptor could be enhanced. Yet, when one treats the cancer patient with an IC blocker based on the ligand (e.g. anti PDL1, that is approved clinically), then it is impossible to enhance the function of effector cells since their PD1 is interacting with PDL2 (the other ligand to PD1) expressed by the cancer cells. In this case the IcAR-PD1 assay is advantageous since it is possible to calculate scores either through anti PD1 used in the assay or through anti PDL1 used in the assay, and thus could indicate in some cases that the patient will better benefit from anti-PD1 treatment and not from anti-PDL1 treatment. For example, if the IcAR-PD1 score is calculated based on anti-PD1 is much higher than the IcAR-PD1 score calculated based on anti-PDL1, then it could indicate that the tumor expresses functional PDL2 that can suppress PD1. FIGS. 6A-B show that indeed the expression of PDL1 and PDL2 varies between PDXs derived from NSCLC and H&N samples (FIG. 6A, 6B). In this case, expression of PDL2 was very low but the present inventors identified cancer cell lines with high expression of PDL2 like A549 (FIG. 6C), and thus it is expected that some cancers will have high levels of PDL-2 that could be also functional inhibitors as was published for colorectal cancers. (Shakerin et al, Mol Biol Rep, 2020 August; 47 (8):5689-5697. doi: 10.1007/s11033-020-05289-7. Epub 2020 Jul. 13).

To summarize, the presence of more than one ligand to the IC receptor (E.g. PDL1 and PDL2 ligands for the IC receptor PD1), can make difference in the therapeutic results with anti PDL1 vs. Anti PD1 therapy; the difference between the IcAR score evaluated with anti-PD1 vs the score evaluated with anti-PDL1 can hint the physician to the presence of additional ligands (eg PDL2) that will reduce the therapeutic potential of anti PDL1 therapy.

### EXAMPLE 8

### Other IcARs are Responding to Clinical Samples Differently from IcAR-PD1 Response—Predicting Alternative Immunotherapy Regimens

IcAR-CTLA4 was produced as described above for cloning and cellular reporter production.

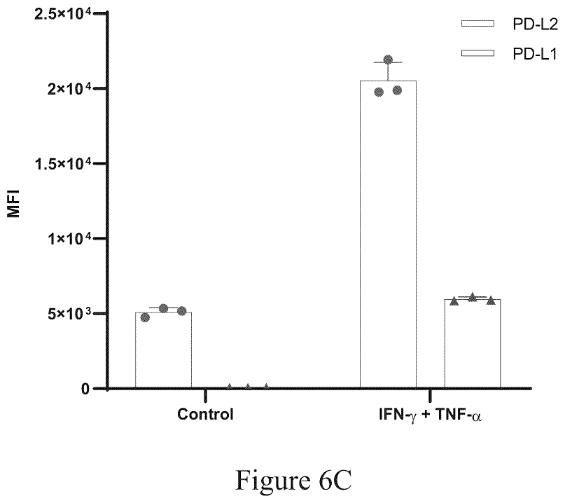

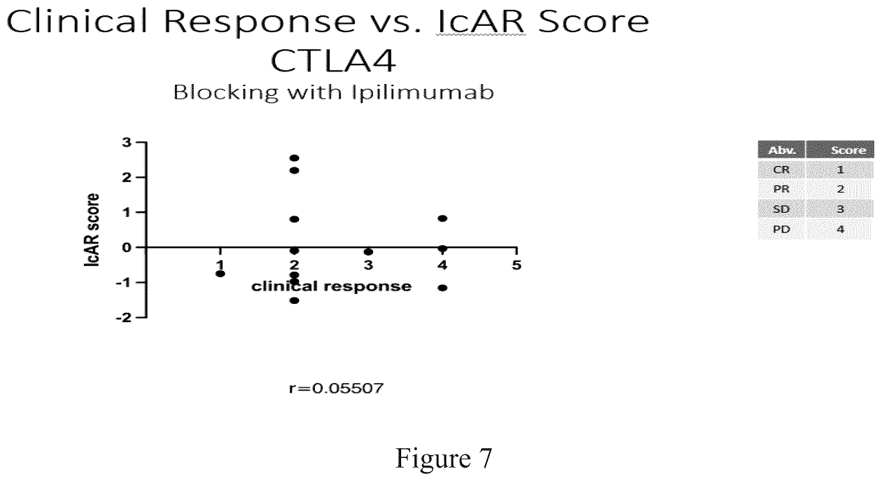

Then the IcAR assay was performed with IcAR-CTLA4 while employing ipilimumab as the drug in the assay. The FFPE samples used were those described in Example 6 (i.e., from lung lesions of cancer patients that were then clinically treated with anti PD1). The IcAR-CTLA4 score was compared to the clinical results of treating with anti PD1. In contrast to the significant high correlation of IcAR-PD1 score with the anti-PD1 clinical results (Example 5), no significant correlation was shown between IcAR-CTLA4 and anti PD1 clinical results (FIG. 7). It is already known that various cancers and cancer patients can respond differently to different ICI therapies and the results from example 6 and FIG. 7 prove that the predictions of the IcAR scoring profile are following the same pattern. Note that patients that had complete clinical response (CR) to anti PD1 therapy and scored highly with the IcAR-PD1 (Example 5) were scored negative with IcAR-CTLA4. These results indicate that the IcAR assay can correctly predict response to an anti PD1. On the other hand, some high scores for with IcAR-PD1 were found in two patients that responded only partially to anti PD1 therapy, indicating that IcAR scoring favors in this case combination of anti PD1 and anti CTLA4 immunotherapy. Note that the IcAR scoring is dependent on the nature of the IC molecules in the chimeric protein, hence PD-1 IcAR has different score than CTLA-4 IcAR. In general, the abundance of ligands to the different IC receptors varies between patients. FIG. 8 shows the expression on PDX of ligands to the IC receptors TIGIT, TIM3, CTLA4 and LAG3 which can be compared to the expression of PDL1 and PDL2 on the same PDXs (FIGS. 6A-C). This staining shows some of the ligands by not their functionality (as the IcAR methodology detect), but it still shows the diverse expression level of the different ligands to different IC receptors indicating the need for IcAR methodology to profile all functional ligands for the different IC receptors.

### EXAMPLE 9

### Testing Matching Normal Tissue with IcAR Add Additional Value to IcAR Prediction (Shown for Colon Adenocarcinoma)

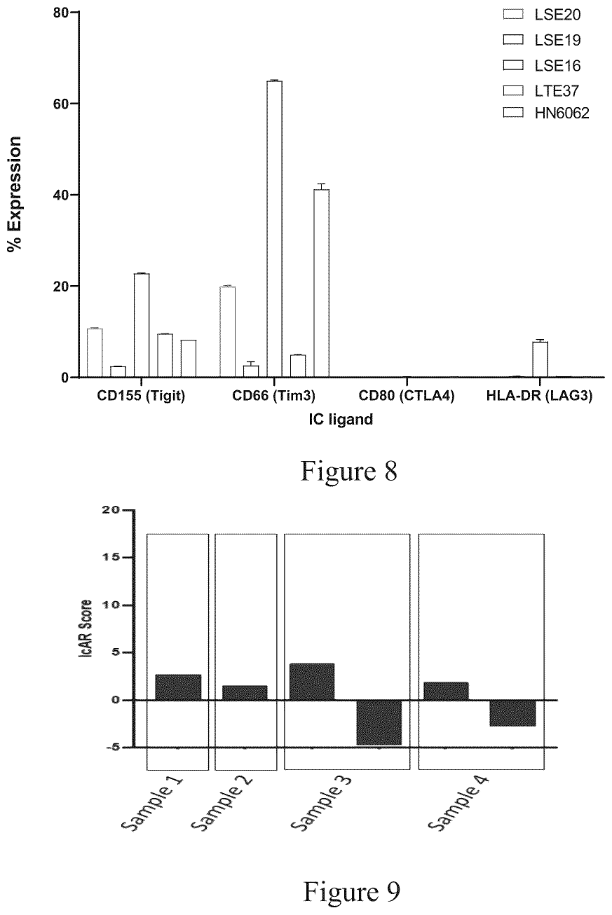

The IcAR assay was applied on 5 micron cuts taken from FFPE blocks of human colon adenocarcinoma and in some cases also on cuts taken from a matching normal tissue. Following the generation of the FFPE samples, patients began immunotherapy with anti PD1. The IcAR assay was performed with the artificial reporter IcAR-PD1 and employed in the assay the anti PD1 drug Keytruda. When a normal matching tissue was available (2 of 4 cases), the same assay was done on cuts from FFPE blocks containing normal matching tissue (FIG. 9), i.e., “control”. The two normal tissues scored negatively with IcAR-PD1 and this was the case for all normal tissues that were tested with IcAR-PD1. However, some differences were observed between the negative scores of normal tissues indicating endogenic usage of PD1 immune checkpoint in non-cancer-related immune responses. Therefore, when taking into account the two scores of cancer and matching normal tissues and putting them in one formula (Cancer score—matching normal score), additional layer of accuracy is adding taking into account the endogenous sensitivity of the patient to the PD1 immune checkpoint. This can also indicate the level of toxicity expected in patients treated with the inhibitor.

### EXAMPLE 10

### IcAR-Tigit Reporter Responses to Clinical Samples

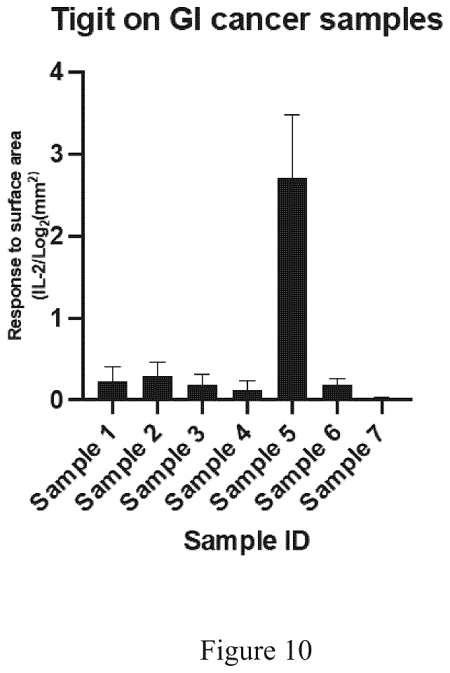

IcAR-Tigit was produced as described above for cloning and cellular reporter production. Then response intensity of the IcAR-Tigit reporter was tested on clinical samples from colon adenocarcinoma. Some of the FFPE tumor samples used were those described in Example 9 (i.e., from colon adenocarcinoma of cancer patients that were then clinically treated with anti PD1). The present inventors tested the intensity of response of the IcAR-Tigit reporter by IL-2. FIG. 10 shows that Tigit-IcAR reporter responded significantly to only one sample (FIG. 10, sample 5) and did not respond to the other colon adenocarcinoma clinical samples. This re-emphasizes the need to profile samples with the various IcAR reporters and to supply the physician with a full map of the responses, so that an educated decision can be made regarding treatment of a specific patient.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A polynucleotide encoding a chimeric polypeptide comprising an amino acid sequence of an immune checkpoint molecule capable of binding a ligand thereof, said immune checkpoint molecule being translationally fused to another amino acid sequence of a cell signaling module such that upon binding of the immune checkpoint molecule to said ligand, said cell signaling module is activated.

2. A polynucleotide encoding a chimeric polypeptide comprising an amino acid sequence of an immune checkpoint molecule capable of binding a receptor thereof, said immune checkpoint molecule being translationally fused to another amino acid sequence of a cell signaling module such that upon binding of the immune checkpoint molecule to said receptor, said cell signaling module is activated.

3. A reporter cell comprising the polynucleotide of claim 1 under transcriptional control of a cis-acting regulatory element(s).

4. A reporter cell comprising the polynucleotide of claim 2.

5. A method of detecting presence and/or activity of a ligand of an immune checkpoint molecule in a cancer cell or a cell in a microenvironment of the cancer cell, the method comprising:
(a) contacting the cancer cell with a reporter cell of claim 3;
(b) determining activation of said cell signaling module in the reporter cell, said activation being indicative of the presence and/or activity of the ligand of the immune checkpoint molecule in the cancer cell or cell in the microenvironment.

6. A method of detecting presence and/or activity of a receptor of an immune checkpoint molecule in an immune cell, the method comprising:
(a) contacting the immune cell with the reporter cell of claim 4;
(b) determining activation of said cell signaling module in the reporter cell, said activation being indicative of the presence and/or activity of the receptor of the immune checkpoint molecule in the immune cell.

7. A method of treating a subject diagnosed with cancer, the method comprising:
(a) contacting the cancer cell or a cell in a microenvironment of the cancer cell of the subject with the reporter cell of claim 3;
(b) determining activation of said cell signaling module in the reporter cell, said activation being indicative of the presence and/or activity of the ligand of the immune checkpoint molecule in the cancer cell or the cell in the microenvironment of the cancer cell; and
(c) treating the subject with a modulator of the immune checkpoint molecule when presence or a predetermined threshold of activity of said ligand of the immune checkpoint molecule is indicated or with another treatment modality when it is not indicated or absent.

8. The chimeric polypeptide encoded by the polynucleotide of claim 1.

9. The method of claim 5, wherein said immune checkpoint molecule is selected from the group consisting of CTLA4, PD-1, LAG3, TIGIT, TIM3, VISTA, CEACAM1, CD28, OX40, CD137(4-1BB), GITR, ICOS, CD27, CD80, CD86, PD-L1, PD-L2, MHC class II/lectins, CD155, Galectin 9, VSIG-3, B7, CD80, CD86, OX40L, CD137L, GITRL, ICOSLG and CD70.

10. The method of claim 5, wherein said immune checkpoint molecule is PD-1 or CTLA4.

11. The method of claim 5, wherein said immune checkpoint molecule is naturally expressed on an immune cell and wherein said ligand is naturally expressed on a cancer cell.

12. The method of claim 5, wherein said cell signaling module comprises a transmembrane domain and/or a cytoplasmic portion of a cell signaling receptor.

13. The method of claim 12, wherein said cell signaling module comprises a transmembrane domain and/or a cytoplasmic portion of a receptor kinase.

14. The method of claim 13, wherein said receptor kinase is a tyrosine kinase or serine/threonine kinase.

15. The method of claim 5, wherein said cell signaling module comprises a CD3 zeta chain.

16. The method of claim 5, wherein activation of said cell signaling module is by dimerization, oligomerization and/or post-translational modification.

17. The method of claim 5, wherein said determining activation is by analyzing a cytokine and/or an interleukin induced by said activation.

18. The method of claim 17, wherein said interleukin is selected from the group consisting of IL-2 and IL-8.

19. The cell of claim 3, transformed to express a fluorescent or bioluminescent molecule upon activation of said cell signaling module.

20. The method of claim 5, wherein said contacting is in the presence of an immune checkpoint modulator.


SEQUENCE LISTING (41 sequences)

SEQ ID NO: 1 - protein, 5 aa, synthetic construct
  REGION 1..5: note = linker amino acid sequence
GGGGS

SEQ ID NO: 2 - protein, 20 aa, synthetic construct
  REGION 1..20: note = linker amino acid sequence
GGGGSGGGGSGGGGSGGGGS

SEQ ID NO: 3 - protein, 9 aa, synthetic construct
  REGION 1..9: note = linker amino acid sequence
GGGGSGGGG

SEQ ID NO: 4 - protein, 10 aa, synthetic construct
  REGION 1..10: note = linker amino acid sequence
GGGGSGGGGS

SEQ ID NO: 5 - protein, 14 aa, synthetic construct
  REGION 1..14: note = linker amino acid sequence
GGGGSGGGGSGGGG

SEQ ID NO: 6 - protein, 8 aa, synthetic construct
  REGION 1..8: note = linker amino acid sequence
GGGGGGGG

SEQ ID NO: 7 - protein, 6 aa, synthetic construct
  REGION 1..6: note = linker amino acid sequence
GGGGGG

SEQ ID NO: 8 - protein, 15 aa, synthetic construct
  REGION 1..15: note = linker amino acid sequence
EAAAKEAAAKEAAAK

SEQ ID NO: 9 - protein, 27 aa, synthetic construct
  REGION 1..27: note = linker amino acid sequence
AEAAAKEAAAKEAAAKEAAAKEAAAKA

SEQ ID NO: 10 - protein, 12 aa, synthetic construct
  REGION 1..12: note = linker amino acid sequence
AEAAAKEAAAKA

SEQ ID NO: 11 - protein, 46 aa, synthetic construct
  REGION 1..46: note = linker amino acid sequence
AEAAAKEAAAKEAAAKEAAAKALEAEAAAKEAAAKEAAAKEAAAKA

SEQ ID NO: 12 - protein, 5 aa, synthetic construct
  REGION 1..5: note = linker amino acid sequence
PAPAP

SEQ ID NO: 13 - protein, 18 aa, synthetic construct
  REGION 1..18: note = linker amino acid sequence
KESGSVSSEQLAQFRSLD

SEQ ID NO: 14 - protein, 14 aa, synthetic construct
  REGION 1..14: note = linker amino acid sequence
EGKSSGSGSESKST

SEQ ID NO: 15 - protein, 12 aa, synthetic construct
  REGION 1..12: note = linker amino acid sequence
GSAGSAAGSGEF

SEQ ID NO: 16 - protein, 134 aa, synthetic construct
  REGION 1..134: note = chimeric polypeptides
LCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 17 - DNA, 405 bp, synthetic construct
  misc_feature 1..405: note = chimeric polypeptides nucleic acid sequence
  source 1..405: mol_type = other DNA
ctctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 18 - DNA, 927 bp, synthetic construct
  misc_feature 1..927: note = chimeric polypeptides nucleic acid sequence
  source 1..927: mol_type = other DNA
gaattcatgcagattccgcaggcgccgtggccggtggtgtgggcggtgctgcagctgggctggcgcccgggctggtttctggatagcccggatcgcccgtggaacccgccgacctttagcccggcgctgctggtggtgaccgaaggcgataacgcgacctttacctgcagctttagcaacaccagcgaaagctttgtgctgaactggtatcgcatgagcccgagcaaccagaccgataaactggcggcgtttccggaagatcgcagccagccgggccaggattgccgctttcgcgtgacccagctgccgaacggccgcgattttcatatgagcgtggtgcgcgcgcgccgcaacgatagcggcacctatctgtgcggcgcgattagcctggcgccgaaagcgcagattaaagaaagcctgcgcgcggaactgcgcgtgaccgaacgccgcgcggaagtgccgaccgcgcatccgagcccgagcccgcgcccggcgggccagtttcagaccggatccctctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaatctaga

SEQ ID NO: 19 - protein, 308 aa, synthetic construct
  REGION 1..308: note = chimeric polypeptides
EFMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTGSLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRSR

SEQ ID NO: 20 - DNA, 1769 bp, synthetic construct
  misc_feature 1..1769: note = chimeric polypeptides nucleic acid sequence
  source 1..1769: mol_type = other DNA
gcggccgcatgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctgggtccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaaggatcc

SEQ ID NO: 21 - protein, 584 aa, synthetic construct
  REGION 1..584: note = chimeric polypeptides
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 22 - DNA, 1025 bp, synthetic construct
  misc_feature 1..1025: note = chimeric polypeptides nucleic acid sequence
  source 1..1025: mol_type = other DNA
gcggccgcatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgattctagcagacagtgggatctactgctgccgaatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcctctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaaggatcc

SEQ ID NO: 23 - protein, 336 aa, synthetic construct
  REGION 1..336: note = chimeric polypeptides
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 24 - DNA, 902 bp, synthetic construct
  misc_feature 1..902: note = chimeric polypeptides nucleic acid sequence
  source 1..902: mol_type = other DNA
gcggccgcatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacctctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaaggatcc

SEQ ID NO: 25 - protein, 295 aa, synthetic construct
  REGION 1..295: note = chimeric polypeptides
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 26 - DNA, 842 bp, synthetic construct
  misc_feature 1..842: note = chimeric polypeptides nucleic acid sequence
  source 1..842: mol_type = other DNA
gcggccgcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaaggatcc

SEQ ID NO: 27 - protein, 275 aa, synthetic construct
  REGION 1..275: note = chimeric polypeptides
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 28 - DNA, 1442 bp, synthetic construct
  misc_feature 1..1442: note = chimeric polypeptides nucleic acid sequence
  source 1..1442: mol_type = other DNA
gcggccgcatgaaaagccagacccaggtgtttgtgtttctgctgctgtgcgtgagcggcgcgcatggcagcattgtgatgacccagaccccgaaatttctgctggtgagcgcgggcgatcgcgtgaccattacctgcaaagcgagccagagcgtgagcaacgatgtggcgtggtatcagcagaaaccgggccagagcccgaaactgctgatttattatgcgagcaaccgctataccggcgtgccggatcgctttaccggcagcggctatggcaccgattttacctttaccattagcaccgtgcaggcggaagatctggcggtgtatttttgccagcaggattatagcagcccgccgtggacctttggcggcggcaccaaactggaaattggcggcggcggcagcggcggcggcggcagcggcggcggcggcagcggcggcggcggcagccaggtgcagctgaaacagagcggcccgggcctggtgcagccgagccagagcctgagcctgacctgcaccgtgagcggctttagcctgaccagctatggcgtgcattgggtgcgccagccgccgggcaaaggcctggaatggctgggcgtgatttggagcggcggcagcaccgattataacgcggcgtttattagccgcctgagcattcgcaaagataacagcaaaagccaggtgttttttaaaatgaacagcctgcaggcggatgataccgcgatttattattgcgcgcgcacctttaccaccagcaccagcgcgtggtttgcgtattggggccagggcaccctggtgaccgtgagcgcggtcgacgaacagaaactgattagcgaagaagatctggcgctgagcaacagcattatgtattttagccattttgtgccggtgtttctgccggcgaaaccgaccaccaccccggcgccgcgcccgccgaccccggcgccgaccattgcgagccagccgctgagcctgcgcccggaagcgagccgcccggcggcgggcggcgcggtgcatacccgcggcctggatctctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaaggatcc

SEQ ID NO: 29 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 30 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPPQAQSKIEELEQEPEPEPEPEPEPEPEPEPEQL

SEQ ID NO: 31 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 32 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 33 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 34 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPMLPNDNGSYRCSANFQSNLIESHSTTLYVTDVKGASERPSKDEVASRPWLLYSLLPLGGLPLLITTWFCLFCCLRRHQGKQNELSDTAGREINLVDAHLKSEQTEASTRQNSQVLLSEAGIYDNDPDLCFRMQEGSEVCSNPCLEENKPGIVYASLNHSVIGLNSRLARNVKEAPTEYASICVRS

SEQ ID NO: 35 - protein, 569 aa, Homo sapiens
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 36 - protein, 5 aa, synthetic construct
  REGION 1..5: note = partial sequence of BTLA
WLLYR

SEQ ID NO: 37 - protein, 15 aa, synthetic construct
  REGION 1..15: note = linker amino acid sequence
GGGGSGGGGSGGGGS

SEQ ID NO: 38 - protein, 10 aa, synthetic construct
  REGION 1..10: note = linker amino acid sequence
EAAAKEAAAK

SEQ ID NO: 39 - protein, 5 aa, synthetic construct
  REGION 1..5: note = linker amino acid sequence
EAAAK

SEQ ID NO: 40 - protein, 22 aa, synthetic construct
  REGION 1..22: note = linker amino acid sequence
AEAAAKEAAAKEAAAKEAAAKA

SEQ ID NO: 41 - protein, 17 aa, synthetic construct
  REGION 1..17: note = linker amino acid sequence
AEAAAKEAAAKEAAAKA